(19) United States
(12) Patent Application Publication
(10) Pub. No.: US 2024/0410805 A1
(43) Pub. Date: Dec. 12, 2024

(54) Method for Measuring Easily Extrusion-Moldable PCR Resin and PCR Resin Composition

(71) Applicants: SK Innovation Co., Ltd., Seoul (KR); SK Geo Centric Co., Ltd., Seoul (KR)

(72) Inventors: Il Young Kwon, Daejeon (KR); Tae Ho Kim, Daejeon (KR); Hak Bin Kim, Daejeon (KR); Si Uk Cheon, Daejeon (KR)

(21) Appl. No.: 18/632,686

(22) Filed: Apr. 11, 2024

(30) Foreign Application Priority Data
Jun. 7, 2023 (KR) 10-2023-0072696

(51) Int. Cl. G01N 11/08; G01N 33/44

(52) U.S. Cl. CPC G01N 11/08; G01N 33/442

(57) ABSTRACT

Provided is a method for determining whether a post-consumer recycled (PCR) resin is an easily extrudable PCR resin by adding the PCR resin to a capillary rheometer and quantifying a volume value measured when a pressure is increased to a certain level, which is a method for providing determination information of the easily extrudable PCR resin.

### CROSS-REFERENCE TO RELATED APPLICATION

This application claims priority to Korean Patent Application No. 10-2023-0072696 filed Jun. 7, 2023, the disclosure of which is hereby incorporated by reference in its entirety.

### BACKGROUND OF THE INVENTION

### Field of the Invention

The present disclosure relates to a method for measuring easily extrudable PCR resin and PCR resin composition.

### Description of Related Art

A post-consumer recycled (PCR) resin refers to a recycled resin, and a study for recycling it is currently in progress. The PCR resin is a polymer resin, such as polyethylene (PE), polyethylene terephthalate (PET), polypropylene (PP), poly(vinyl chloride) (PVC), and/or polystyrene (PS), and the polymer resin is mainly used in clothing, carpets, bottles, plastic lumber, pipes, floor tiles, packaging, fibers, and/or the like, and thus, the content of a discharged PCR resin is increasing every year.

Among the PCR resin materials, in the case of stretch wrap used to package large supermarket items of a polyethylene (PE) resin, resin having a large volume and the same properties may be secured in a large amount, and thus, a study for recycling the resin continues.

However, since a polyethylene PCR resin usually has deteriorated physical properties and processability as compared with a new material polymer resin, it is difficult to use the polyethylene PCR resin again. In particular, the physical properties are greatly deteriorated due to foreign matter inside the resin of the polyethylene PCR resin after processing, and in particular, even when a foreign matter removal filter is separately provided during extrusion, the filter in an extruder is eventually blocked by excessive foreign matter during processing, so that continuous processing is very difficult.

Accordingly, the collected polyethylene PCR resin materials should be differentiated and classified into recyclable grade A resins, non-recyclable C grade resins, and the like, but it is difficult to classify the grade A PCR resin and the grade C PCR resin by visual inspection, ICP elemental analysis, TGA pyrolysis analysis, and the like, and the cost of the analysis is also high.

### SUMMARY OF THE INVENTION

In some embodiments of the present disclosure, there is provided a method for determining an easily extrudable PCR resin which enables easy classification of a PCR resin comprising a foreign matter content at or below a certain level by using a capillary rheometer device of a grade A PCR resin material having a low content of foreign matter.

In some embodiments of the present disclosure, there is provided a PCR resin composition which is easily processed by mixing a low-grade PCR resin with a new material at an optimal content, since a composition of a mixture of the PCR resin and the new material is measured using a capillary rheometer device to determine whether the PCR resin composition is an easily extrudable PCR resin.

In some embodiments, a method for determining whether a post-consumer recycled (PCR) resin is an easily extrudable post-consumer recycled (PCR) resin is provided, wherein the PCR resin comprises a polyolefin resin, the method comprising: a) passing the PCR resin through a capillary rheometer to determine whether: (1) a total capacity of the PCR resin at which an initial pressure (P₀) becomes doubled (Pₜ) when the PCR resin passes through a capillary rheometer satisfies the following Equation 1, (2) a ratio between a piston diameter and a capillary diameter in the capillary rheometer satisfies the following Equation 2, and (3) a sieve mesh size and a wire diameter of a mesh included in the capillary rheometer satisfy the following Equation 3:

$$\frac{( {V}_{t} - {V}_{a} )}{{V}_{t}} < 0.8 \qquad [ Equation 1 ]$$

wherein Vₜ is an initial volume (cm³) of the PCR resin entering the capillary rheometer, and Vₐ is a volume (cm³) of a resin filtered by the mesh in the capillary rheometer at which the initial pressure becomes doubled,

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm), and
b) determining whether the PCR resin passing through the capillary rheometer satisfies each of Equations 1, 2 and 3, and if so then the PCR resin is an easily extrudable post-consumer recycled (PCR) resin.

In some embodiments, the PCR resin may comprise a polyethylene resin.

In some embodiments, the mesh comprised in the capillary rheometer may be 50 to 200 meshes.

In some embodiments, the mesh may have a total permeation area ratio of 30 to 50%.

In some embodiments, a pressure applied to the capillary rheometer may be 10 to 1,000 bar.

In some embodiments, a cylinder internal temperature of the capillary rheometer may satisfy the following Equation 4:

$$P {T}_{1} + 50 ° C . < {T}_{1} < P {T}_{1} + 150 ° C . \qquad [ Equation 4 ]$$

wherein PT₁ is a melting temperature of the PCR resin, and T₁ is the cylinder internal temperature of the capillary rheometer.

In some embodiments, the capillary rheometer may have the piston diameter of 10 to 30 mm and the capillary diameter of 1 to 5 mm.

In some embodiments, a method for determining whether a post-consumer recycled (PCR) resin is an easily extrudable post-consumer recycled (PCR) resin, wherein the PCR resin comprises a polyolefin resin, the method comprising: a) passing the PCR resin through a capillary rheometer to determine whether: (1) the following Equation 5 is satisfied when the PCR resin passes through a capillary rheometer, (2) a ratio between a piston diameter and a capillary diameter in the capillary rheometer satisfies the following Equation 2, and (3) a sieve mesh size and a wire diameter of a mesh included in the capillary rheometer satisfy the following Equation 3:

$$\frac{ln ( \frac{{P}_{L}}{{P}_{0}} )}{V} < 0 \text{.16} \qquad [ Equation 5 ]$$

$$0.05 \leq \frac{{M}_{2}}{{M}_{1}} \leq 0.2 \qquad [ Equation 6 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm); and b) determining whether the PCR resin passing through the capillary rheometer satisfies each of Equations 1, 2 and 3, and if so then the PCR resin is an easily extrudable post-consumer recycled (PCR) resin.

P₀ is an initial internal pressure of the capillary rheometer when the PCR resin is added and a pressure was applied at a volume of 1 cc, and P_{L} is a pressure finally applied to the capillary rheometer. V is an internal volume of the capillary rheometer decreased by additional pressure from P₀, and V refers to a volume until a P_{L} value becomes twice a P₀ value. When the P_{L} value is more than twice the P₀ value, V is measured with P_{L} being limited to twice P₀. In some embodiments, when P_{L} is less than twice P₀, V is set as a final measured volume.

In some embodiments, the PCR resin may comprise a new material.

In some embodiments, the mesh comprised in the capillary rheometer may be 50 to 200 meshes.

In some embodiments, the mesh may have a total permeation area ratio of 35 to 50%.

In some embodiments, a cylinder internal temperature of the capillary rheometer satisfies the following Equation 4:

$$P {T}_{1} + 50 ° C . < {T}_{1} < P {T}_{1} + 150 ° C . \qquad [ Equation 4 ]$$

wherein PT₁ is a melting temperature of the PCR resin, and T₁ is the cylinder internal temperature of the capillary rheometer.

In some embodiments, the capillary rheometer may have the piston diameter of 10 to 30 mm and the capillary diameter of 1 to 5 mm.

In some embodiments, an excellently melt-extrudable post-consumer recycled (PCR) resin is provided, wherein a total capacity of a resin at which an initial pressure (P₀) becomes doubled (Pₜ) when the PCR resin passes through a capillary rheometer satisfies the following Equation 1, a ratio between a piston diameter and a capillary diameter in the capillary rheometer satisfies the following Equation 2, and a sieve mesh size and a wire diameter of a mesh included in the capillary rheometer satisfy the following Equation 3:

$$\frac{( {V}_{t} - {V}_{a} )}{{V}_{t}} < 0.8 \qquad [ Equation 1 ]$$

wherein Vₜ is an initial volume (cm³) of the PCR resin entering the capillary rheometer, and Vₐ is a volume (cm³) of a resin filtered by the mesh in the capillary rheometer at which the initial pressure becomes doubled,

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

In some embodiments, a post-consumer recycled (PCR) resin composition is provided, wherein the PCR resin comprises a polyolefin resin, the following Equation 5 is satisfied when the PCR resin passes through a capillary rheometer, a ratio between a piston diameter and a capillary diameter in the capillary rheometer satisfies the following Equation 2, and a sieve mesh size and a wire diameter of a mesh included in the capillary rheometer satisfy the following Equation 3:

$$\frac{ln ( \frac{{P}_{L}}{{P}_{0}} )}{V} < 0 \text{.16} \qquad [ Equation 5 ]$$

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

P₀ is an initial internal pressure of the capillary rheometer when the PCR resin is added and a pressure was applied at a volume of 1 cc, and P_{L} is a pressure finally applied to the capillary rheometer. In addition, V is an internal volume of the capillary rheometer decreased by additional pressure from P₀, and V refers to a volume until a P_{L} value becomes twice a P₀ value. When the P_{L} value is more than twice the P₀ value, V is measured with P_{L} being limited to twice P₀. In some embodiments, when P_{L} is less than twice P₀, V is set as a final measured volume.

In some embodiments, the PCR resin composition may comprise a new material.

Other features and aspects will be apparent from the following detailed description, the drawings, and the claims.

### BRIEF DESCRIPTION OF THE DRAWINGS

FIG. 1 is a rough schematic diagram of a capillary rheometer device.

FIG. 2 is a graph in which a PCR resin volume to reach an optional pressure using the capillary rheometer device is schematized.

FIG. 3 is a graph of measuring a pressure state occurring when a piston is pressed at a constant speed in the capillary rheometer device.

FIG. 4 is a graph showing a value depending on a PCR resin content in a PCR resin composition using the capillary rheometer device.

### DETAILED DESCRIPTION OF MAIN ELEMENTS

1000: Capillary rheometer device
100: Cylinder body
200: Piston
300: Pressure gauge
400: Mesh
500: Capillary die

### DESCRIPTION OF THE INVENTION

Hereinafter, the present disclosure will be described in more detail with reference to specific examples and exemplary embodiments including the accompanying drawings. However, the following specific examples and exemplary embodiments are only a reference for describing the present disclosure in detail, and the present disclosure is not limited thereto and may be implemented in various forms.

Unless otherwise defined, all technical terms and scientific terms have the same meanings as those commonly understood by one of those skilled in the art to which the present disclosure pertains. The terms used herein are only for effectively describing a certain specific example, and are not intended to limit the present disclosure.

Unless the context clearly indicates otherwise, the singular form used in the specification and claims appended thereto may be intended to include a plural form also, unless otherwise indicated in the context. As used herein, the singular form of “a”, “an”, and “the” include plural referents unless the context clearly states otherwise.

The numerical range used in the present specification comprises all values within the range comprising the lower limit and the upper limit, increments logically derived in a form and spanning in a defined range, all double limited values, and all possible combinations of the upper limit and the lower limit in the numerical range defined in different forms. As an example, when it is defined that a content of a composition is 10% to 80% or 20% to 50%, it should be interpreted that a numerical range of 10% to 50% or 50% to 80% is also described in the specification of the present. Unless otherwise defined in the present specification, values which may be outside a numerical range due to experimental error or rounding off of a value are also comprised in the defined numerical range.

For the purposes of this specification, unless otherwise indicated, all numbers expressing quantities of ingredients, reaction conditions, dimensions, physical characteristics, and so forth used in the specification and claims are to be understood as being modified in all instances by the term “about.” Hereinafter, unless otherwise particularly defined in the present specification, “about” may be considered as a value within 30%, 25%, 20%, 15%, 10%, 5%, 3%, 2%, 1%, 0.5%, 0.1%, 0.05% or 0.01 of a stated value. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Unless particularly described to the contrary, “comprising”, “including”, “having” or “containing” any elements will be understood to imply further inclusion of other elements rather than the exclusion of any other elements.

Conventionally collected polyethylene PCR resin materials should be differentiated and classified into recyclable grade A resins, non-recyclable C grade resins, and the like, but it is difficult to classify grade A PCR resin and grade C PCR resin materials by visual inspection, ICP elemental analysis, TGA pyrolysis analysis, and the like, and the cost of the analysis is also high.

Accordingly, in some embodiments, the present disclosure provides a method for determining whether a post-consumer recycled (PCR) resin is an easily extrudable post-consumer recycled (PCR) resin, wherein the PCR resin comprises a polyolefin resin, the method comprising: a) passing the PCR resin through a capillary rheometer to determine whether: (1) a total capacity of a resin at which an initial pressure (P₀) becomes doubled (Pₜ) when the PCR resin passes through a capillary rheometer satisfies the following Equation 1, (2) a ratio between the piston diameter and the capillary diameter in the capillary rheometer satisfies the following Equation 2, and (3) a sieve mesh size and a wire diameter of a mesh included in the capillary rheometer satisfy the following Equation 3; and b) determining whether the PCR resin passing through the capillary rheometer satisfies each of Equations 1, 2 and 3, and if so then the PCR resin is an easily extrudable post-consumer recycled (PCR) resin.

In some embodiments, the polyolefin resin may comprise or be any one or more polyolefin resins selected from polyethylene, polypropylene, polyketone, polypropylene copolymer, polymethylpentene, polystyrene, and/or polyvinyl chloride, or any one or more polyolefin resins selected from polyethylene, polypropylene, and/or polystyrene, or a polyethylene resin, but is not limited thereto.

According to some embodiments of the present disclosure, the polyethylene resin may comprise or be any one or more polyethylene resins selected from low density polyethylene (LDPE), linear low density polyethylene (LLDPE), crosslinked high density polyethylene (XLPE), and/or high density polyethylene, but is not limited thereto:

$$\frac{( {V}_{t} - {V}_{a} )}{{V}_{t}} < 0.8 \qquad [ Equation 1 ]$$

wherein Vₜ is an initial volume (cm³) of the PCR resin entering the capillary rheometer, Vₐ is a volume (cm³) of a resin filtered by the mesh from the capillary rheometer at which the initial pressure becomes doubled, and when all resin inside the capillary rheometer is discharged without reaching twice the pressure, a Vₐ value is set as a Vₜ,

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

A high-quality continuously extrudable PCR resin material may be easily determined without particular analysis and device in the collected PCR resin, by the method for measuring an easily extrudable PCR resin disclosed herein.

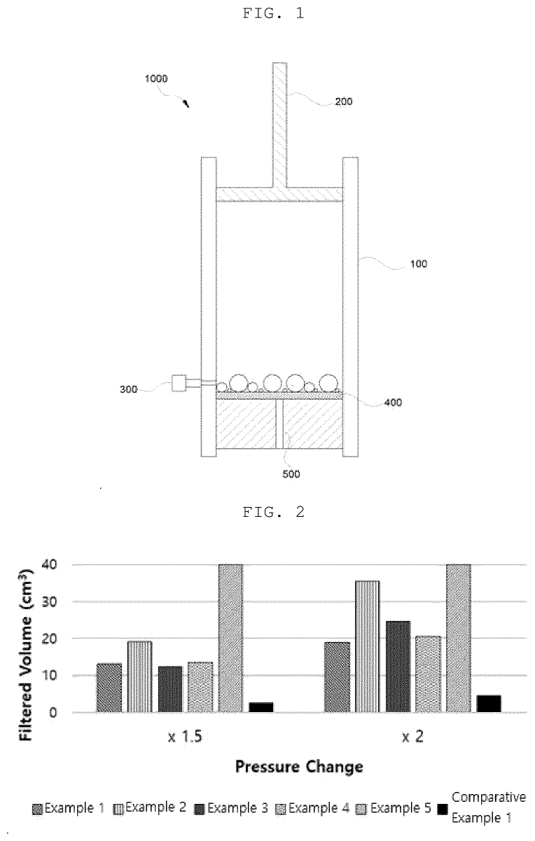

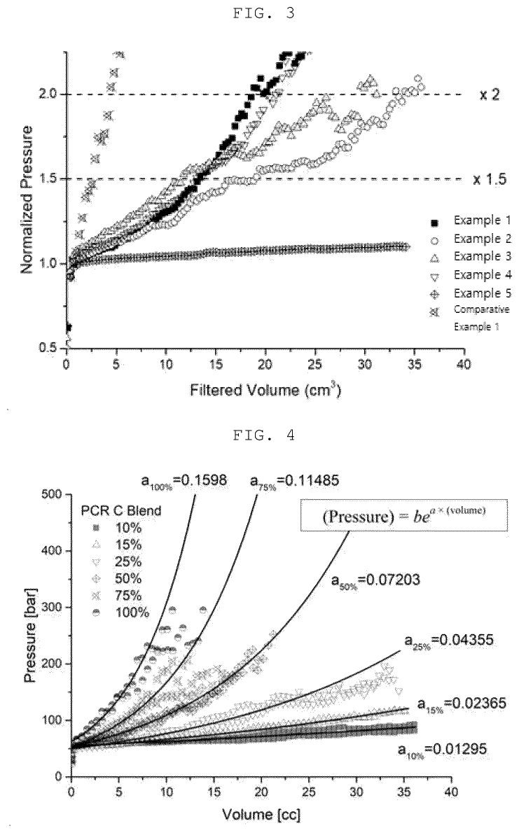

Now, the method of determining whether a post-consumer recycled (PCR) resin is an easily extrudable PCR resin will be described using FIG. 1 as an example. However, FIG. 1 is only an example for describing the capillary rheometer of the present disclosure in detail, and the present disclosure is not limited to FIG. 1.

The capillary rheometer device 1000 comprises a cylinder body 100, a piston 200, a pressure gauge 300, a mesh 400, and a capillary die 500, but the constituents may be further added, if necessary.

A PCR resin material is added to the capillary rheometer device 1000, the cylinder body 100 is heated until the PCR resin material is melted, and then the piston 200 is pressed at a certain speed to discharge a melted PCR resin through the capillary of the capillary die 500.

The certain speed of the piston may be 0.01 to 0.5 mmsec⁻¹, 0.05 to 0.3 mmsec⁻¹, or 0.08 to 0.2 mmsec⁻¹ but is not limited thereto.

Since the mesh 400 is installed in a lower portion of the cylinder body 100, when the melted PCR resin is discharged, foreign matter inside the PCR resin blocks the hole of the mesh and the internal pressure of the cylinder body 100 is increased. Herein, as the foreign matter content is high, the pressure is rapidly increased even when the piston 200 is pressed a little, and when the pressure is excessively applied, the mesh may burst.

That is, in the present disclosure, a continuously extrudable PCR resin may be distinguished by calculating a time taken to reach a certain pressure or a PCR resin volume inside the cylinder body, depending on the content of foreign matter.

When the PCR resin satisfying the following Equation 1 of the present disclosure is used, it is continuously extrudable and a PCR resin having excellent physical properties may be distinguished. In some embodiments, in the case of a PCR resin having a value depending on a pressure in the following Equation 1 of 0.8 or more, a mesh for filtering foreign matter in extrusion in an extruder may be broken due to excessive pressure, and even when a film is manufactured by extrusion, the physical properties of the film may be greatly deteriorated due to excessive foreign matter. However, in the case of a PCR resin having a value depending on the pressure in the following Equation 1 of less than 0.8, extrusion is well performed, and when a film is manufactured, film manufacture by excessive foreign matter due to an excessive amount is suppressed, and thus, mechanical properties such as tensile strength are excellent. The following Equation 1 may be less than 0.8, or the upper limit may be 0.7 or less, 0.6 or less, 0.5 or less, 0.4 or less, 0.3 or less, 0.2 or less, or 0.1 or less and the lower limit may be 0.0 or more, 0.001 or more, and, 0.0 or more and less than 0.8, or 0.001 or more and less than 0.8, or 0.01 to 0.7, but the present disclosure is not limited thereto.

As the following Equation 1 is close to 0, the foreign matter content is almost 0, and when the following Vₜ value and the Vₐ value are the same, it may have equivalent quality to a new material having almost no foreign matter:

$$\frac{( {V}_{t} - {V}_{a} )}{{V}_{t}} < 0.8 \qquad [ Equation 1 ]$$

wherein Vₜ is an initial volume (cm³) of the PCR resin entering the capillary rheometer, Vₐ is a volume (cm³) of a resin filtered by the mesh from the capillary rheometer at which the initial pressure becomes doubled, and when all resin inside the capillary rheometer is filtered without reaching twice the pressure, a Vₐ value is set as a Vₜ.

A standard of the capillary rheometer device 1000 from which Equation 1 is derived may be a device satisfying the following Equations 2 and 3:

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

In some embodiments, a PCR resin satisfying Equation 1 using the capillary rheometer device 1000 in which a piston diameter and a capillary diameter of the capillary rheometer device 1000 satisfy Equation 2 and a wire diameter of the mesh 400 and a sieve mesh size are based on Equation 3 has excellent continuous extrudability and when it is processed into a film, its physical properties, such as tensile strength, are also excellent.

According to some embodiments of the present disclosure, the PCR resin may comprise a polyethylene resin, and since the polyethylene resin PCR resin which is distinguished as a PCR resin satisfying Equation 1 using the capillary rheometer device 1000 satisfying Equations 2 and 3 is extrusion-molded, a polyethylene PCR resin product which has excellent physical properties and is continuously extrudable without blockage may be provided.

According to some embodiments of the present disclosure, the mesh comprised in the capillary rheometer may have an upper limit of 200 meshes or less and a lower limit of 50 meshes or more, or 50 to 200 meshes, but is not limited thereto.

According some embodiments of the present disclosure, the mesh may have a total permeation area ratio of 30 to 50%, 50% or less, 48% or less, 45% or less, 43% or less, 42% or less, 41% or less, or 40% or less as an upper limit and 30% or more, 33% or more, 36% or more, 37% or more, 38% or more, or 39% or more as a lower limit, 37 to 48%, 38 to 40%, but is not limited thereto.

According to some embodiments of the present disclosure, a pressure applied to the capillary rheometer may be 10 to 1,000 bar, or 10 to 500 bar, or 30 to 100 bar, but is not limited thereto.

According to some embodiments of the present disclosure, a cylinder internal temperature of the capillary rheometer may satisfy the following Equation 4, but it is not limited thereto as long as it is in a melting temperature range of a polymer, and as an example, may be 300° C. or lower, 250° C. or lower, or 200° C. or lower as an upper limit and may be 50° C. or higher, 100° C. or higher, or 150° C. or higher as a lower limit, or 50 to 300° C., 100 to 300° C., 150 to 300° C., 150 to 250° C., or 150 to 230° C., but is not limited thereto:

$${PT}_{1} + 50 ° C . < {T}_{1} < {PT}_{1} + 150 ° C . \qquad [ Equation 4 ]$$

wherein PT₁ is a melting temperature of the PCR resin, and T₁ is the cylinder internal temperature of the capillary rheometer.

According to some embodiments of the present disclosure, the piston diameter of the capillary rheometer may be 10 to 30 mm, or 12 to 25 mm and the capillary diameter may be 1 to 5 mm, or 1.5 to 3 mm, but the present disclosure is not limited thereto.

The present disclosure may separate and provide an excellently melt-extrudable post-consumer recycled (PCR) resin, wherein a total capacity of a resin at which an initial pressure (P0) becomes doubled (Pₜ) when the PCR resin passes through a capillary rheometer satisfies the following Equation 1, a ratio between a piston diameter and a capillary diameter in the capillary rheometer satisfies the following Equation 2, and a sieve mesh size and a wire diameter of a mesh comprised in the capillary rheometer satisfy the following Equation 3. Since the PCR resin having excellent melt extrudability satisfies the following Equation 1, a recycled film having excellent physical properties and also excellent processability may be provided. Since the capillary rheometer is described in detail above, the description thereof will be omitted here:

$$\frac{( {V}_{t} - {V}_{a} )}{{V}_{t}} < 0.8 \qquad [ Equation 1 ]$$

wherein Vₜ is an initial volume (cm³) of the PCR resin entering the capillary rheometer, and Vₐ is a volume (cm³) of a resin filtered by the mesh in the capillary rheometer at which the initial pressure becomes doubled,

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

Since Equations 1 to 3 are described in detail above, the description thereof will be omitted here.

According to some embodiments of the present disclosure, the PCR resin may comprise a polyethylene resin, but since it is also described above, the description thereof will be omitted here.

According to some embodiments of the present disclosure, the mesh comprised in the capillary rheometer may be 50 to 200 meshes, but since it is also described above, the description thereof will be omitted here.

According to some embodiments of the present disclosure, the mesh may have a total permeation area ratio of 35 to 50%, but since it is also described above, the description thereof will be omitted here.

According to some embodiments of the present disclosure, a pressure applied to the capillary rheometer may be 10 to 1,000 bar, but since it is also described above, the description thereof will be omitted here.

According to some embodiments of the present disclosure, a cylinder internal temperature of the capillary rheometer may satisfy the following Equation 4, but since it is also described above, the description thereof will be omitted here:

$${PT}_{1} + 50 ° C . < {T}_{1} < {PT}_{1} + 150 ° C . \qquad [ Equation 4 ]$$

wherein PT₁ is a melting temperature of the PCR resin, and T₁ is the cylinder internal temperature of the capillary rheometer.

According to some embodiments of the present disclosure, the capillary rheometer may have the piston diameter of 10 to 30 mm and the capillary diameter of 1 to 5 mm, but since it is also described above, the description thereof will be omitted here.

The PCR resin having excellent melt extrudability may be used alone by melt extrusion, or may be used with a new material further mixed with the PCR resin.

The new material may be the polyolefin-based resin described above, but is not limited thereto.

According to some embodiments of the present disclosure, an easily extrudable post-consumer recycled (PCR) resin composition may be provided, wherein the PCR resin comprises a polyolefin resin, the following Equation 5 is satisfied when the PCR resin passes through a capillary rheometer, a ratio between a piston diameter and a capillary diameter in the capillary rheometer satisfies the following Equation 2, and a sieve mesh size and a wire diameter of a mesh comprised in the capillary rheometer satisfy the following Equation 3:

$$\frac{ln ( \frac{{P}_{L}}{{P}_{0}} )}{V} < 0 \text{.16} \qquad [ Equation 5 ]$$

$$0.05 \leq \frac{{M}_{2}}{{M}_{1}} \leq 0.2 \qquad [ Equation 2 ]$$

wherein M₁ is a piston diameter, and M₂ is a capillary diameter,

$$0.65 \leq \frac{W D}{S M} \leq 0.73 \qquad [ Equation 3 ]$$

wherein WD is a wire diameter (mm), and SM is a sieve mesh size (mm).

P₀ is an initial internal pressure of the capillary rheometer when the PCR resin is added and a pressure was applied at a volume of 1 cc, and P_{L} is a pressure finally applied to the capillary rheometer. V is an internal volume of the capillary rheometer decreased by additional pressure from PC, and V refers to a volume until a P_{L} value becomes twice a P₀ value. When the P_{L} value is more than twice the P₀ value, V is measured with P_{L} being limited to twice P₀. In addition, when P_{L} is less than twice P₀, V is set as a final measured volume.

Since Equations 2, 3, and 5 are described in detail herein, the description thereof will be omitted here.

According to some embodiments of the present disclosure, the PCR resin may comprise a polyethylene resin, but since it is also described herein, the description thereof will be omitted here.

According to some embodiments of the present disclosure, the mesh comprised in the capillary rheometer may be 50 to 200 meshes, but since it is also described herein, the description thereof will be omitted here.

According to some embodiments of the present disclosure, the mesh may have a total permeation area ratio of 35 to 50%, but since it is also described herein, the description thereof will be omitted here.

According to some embodiments of the present disclosure, a pressure applied to the capillary rheometer may be 10 to 1,000 bar, but since it is also described herein, the description thereof will be omitted here.

According to some embodiments of the present disclosure, a cylinder internal temperature of the capillary rheometer may satisfy the following Equation 4, but since it is also described herein, the description thereof will be omitted here:

$${PT}_{1} + 50 ° C . < {T}_{1} < {PT}_{1} + 150 ° C . \qquad [ Equation 4 ]$$

wherein PT₁ is a melting temperature of the PCR resin, and T₁ is the cylinder internal temperature of the capillary rheometer.

According to some embodiments of the present disclosure, the capillary rheometer may have the piston diameter of 10 to 30 mm and the capillary diameter of 1 to 5 mm, but since it is also described herein, the description thereof will be omitted here.

Hereinafter, the examples of the present disclosure will be further described with reference to the specific experimental examples. It is apparent to those skilled in the art that the examples and the comparative examples included in the experimental examples only illustrate the present disclosure and do not limit the appended claims, and various modifications and alterations of the examples may be made within the range of the scope and spirit of the present invention, and these modifications and alterations will fall within the appended claims.

### Examples and Comparative Examples

An increased pressure occurring in a sample and a flow rate of a sample which passed through a capillary rheometer die were measured using a capillary rheometer (RHEOGRAPH 25, Gottfert).

The measurement was performed using a capillary die having a diameter of 2.0 mm and a length of 25 mm. The diameter of the piston of the rheometer device was 15 mm and a length of 250 mm, and a 120 mesh filter having a sieve size of 0.13 mm, a wire diameter of 0.08 mm, and an opening ratio of 38.3% was mounted on the upper portion of the capillary die.

A capillary die on which a 120 mesh filter was attached was mounted on the lower end of the barrel of the rheometer device and the temperature was maintained at a die test temperature of 190° C. When a PCR resin sample was loaded in a barrel area of the rheometer, air trapped in the sample was all removed so that a volume of 40 cm³ or more was loaded, and then the sample was extruded at a selected piston descending speed of 0.1 mmsec⁻¹ through a capillary die in a barrel. When the sample was processed by passing through a capillary die in the barrel, the pressure applied to the sample and the amount of the sample filtered through the capillary die on which the mesh was mounted were recorded over time to confirm a pressure increase tendency by foreign matter. The results are listed in the following Table 1:

TABLE 1

| | | Example | Example | Example | Example | Example | Comparative |
|---|---|---|---|---|---|---|---|
| | | 1 | 2 | 3 | 4 | 5 | Example 1 |
| Type of resin | | PCR A-1 | PCR A-2 | PCR A-3 | PCR A-4 | PCR A-5 | PCR C |
| Volume | 1.5 times the | 13.13 | 19.20 | 12.26 | 13.40 | ≥40 | 2.50 |
| (cm³) | initial pressure | | | | | | |
| | Later volume/ | 0.672 | 0.52 | 0.694 | 0.665 | 0 | 0.938 |
| | initial volume | | | | | | |
| | 2.0 times the | 18.88 | 35.43 | 24.62 | 20.57 | ≥40 | 4.48 |
| | initial pressure | | | | | | |
| | Later volume/ | 0.528 | 0.114 | 0.385 | 0.486 | 0 | 0.888 |
| | initial volume | | | | | | |

The resins in Table 1 and FIG. 1 are those collected by each company, and as a result of extruding the PCR resin under the capillary rheometer conditions of the present disclosure, all except Comparative Example 1 satisfied the conditions of Equation 1. In FIG. 2, it was confirmed that in Comparative Example 1, when the PCR resin was pushed out at a certain speed, the pressure was rapidly increased.

For the PCR resin composition, a mixed composition of new material polyethylene resin with the PCR resin of the comparative example was added to a capillary rheometer and then the measurement was performed in the same manner as in the comparative example. Content ranges of the new material in the PCR resin are shown in Table 1 and FIG. 4:

TABLE 2

| PCR resin | PCR resin: PCR C | | | | | |
|---|---|---|---|---|---|---|
| content | 10 wt % | 15 wt % | 25 wt % | 50 wt % | 75 wt % | 100 wt % |
| a value | 0.01295 | 0.02365 | 0.04355 | 0.07203 | 0.11485 | 0.1598 |

Each measurement was performed with the capillary rheometer device in a range of 10 to 100 wt % of the PCR resin content in the PCR resin composition, and measurements were substituted into Equation 5 to derive the a value. As shown in Table 2 and FIG. 4, it is confirmed that the value is linearly related to the PCR resin content ratio, and extrusion processing is performed well from the PCR resin content of 75 wt % or less. Thus, it is shown that when a resin is processed, a thin film and the like are manufactured, a PCR resin mixing ratio may be identified in advance through Equation 5.

As a result, in the present disclosure, it was identified that extrusion processing is not performed well due to foreign matter in a PCR resin and a method for separating a PCR resin which is extrusion-processed well, which may identify it in advance, is described, and in particular, even for a low grade PCR resin or a composition mixed with a new material, a method for separating a PCR resin composition which may identify a critical point of a PCR resin content at which extrusion processing is well performed in advance is described.

The method for measuring a PCR resin of the present disclosure may easily determine a continuously extrudable PCR resin material by deriving a correlation between a foreign matter and an extrusion pressure of the PCR resin using a capillary rheometer device.

Also, the present disclosure may provide a PCR resin composition which is easily processed by mixing even a low grade PCR resin with a new material at an optimal content, since a composition of a mixture of a low grade PCR resin and a new material is measured using a capillary rheometer device.

Hereinabove, although the present disclosure has been described by specific matters, limited implementations, and drawings, they have been provided only for assisting the entire understanding of the present disclosure, and the present disclosure is not limited to the implementations, and various modifications and changes may be made by those skilled in the art to which the present disclosure pertains from the description.

Therefore, the spirit of the present disclosure should not be limited to the above-described exemplary embodiments, and the following claims as well as all modified equally or equivalently to the claims are intended to fall within the scope and spirit of the disclosure.

What is claimed is:

1. A method for determining whether a post-consumer recycled (PCR) resin is an easily extrudable post-consumer recycled (PCR) resin, wherein the PCR resin comprises a polyolefin resin, the method comprising:
a) passing the PCR resin through a capillary rheometer to determine whether:
(1) a total capacity of the PCR resin at which an initial pressure (P₀) becomes doubled (Pₜ) when the PCR resin passes through the capillary rheometer satisfies the following Equation 1,
(2) a ratio between a piston diameter and a capillary diameter in the capillary rheometer satisfies the following Equation 2, and
(3) a sieve mesh size and a wire diameter of a mesh included in the capillary rheometer satisfy the following Equation 3:
$$\frac{( {V}_{t} - {V}_{a} )}{{V}_{t}} < 0.8 \qquad [ Equation 1 ]$$
wherein Vₜ is an initial volume (cm³) of the PCR resin entering the capillary rheometer, and
Vₐ is a volume (cm³) of a resin filtered by the mesh in the capillary rheometer at which the initial pressure becomes doubled,
$$0.05 \leq \frac{{M}_{2}}{{M}_{1}} \leq 0.2 \qquad [ Equation 2 ]$$
wherein M₁ is a piston diameter, and M₂ is a capillary diameter,
$$0.65 \leq \frac{W D}{S M} \leq 0.73 \qquad [ Equation 3 ]$$
wherein WD is a wire diameter (mm), and SM is a sieve mesh size (mm), and
b) determining whether the PCR resin passing through the capillary rheometer satisfies each of Equations 1, 2 and 3, and if so then the PCR resin is an easily extrudable post-consumer recycled (PCR) resin.

2. The method for determining whether a post-consumer recycled (PCR) resin is an easily extrudable PCR resin of claim 1, wherein the PCR resin comprises a polyethylene resin.

3. The method for determining whether a post-consumer recycled (PCR) resin is an easily extrudable PCR resin of claim 1, wherein the mesh comprised in the capillary rheometer is 50 to 200 meshes.

4. The method for determining whether a post-consumer recycled (PCR) resin is an easily extrudable PCR resin of claim 3, wherein the mesh has a total permeation area ratio of 30 to 50%.

5. The method for determining whether a post-consumer recycled (PCR) resin is an easily extrudable PCR resin of claim 1, wherein a pressure applied to the capillary rheometer is 10 to 1,000 bar.

6. The method for determining whether a post-consumer recycled (PCR) resin is an easily extrudable PCR resin of claim 1, wherein a cylinder internal temperature of the capillary rheometer satisfies the following Equation 4:
$${PT}_{1} + 50 ° C . < {T}_{1} < {PT}_{1} + 150 ° C . \qquad [ Equation 4 ]$$
wherein PT₁ is a melting temperature of the PCR resin, and T₁ is the cylinder internal temperature of the capillary rheometer.

7. The method for determining whether a post-consumer recycled (PCR) resin is an easily extrudable PCR resin of claim 1, wherein the capillary rheometer has the piston diameter of 10 to 30 mm and the capillary diameter of 1 to 5 mm.

8. A method for determining whether a post-consumer recycled (PCR) resin is an easily extrudable post-consumer recycled (PCR) resin, wherein the PCR resin comprises a polyolefin resin, the method comprising:
a) passing the PCR resin through a capillary rheometer to determine whether:
(1) the following Equation 5 is satisfied when the PCR resin passes through a capillary rheometer,
(2) a ratio between a piston diameter and a capillary diameter in the capillary rheometer satisfies the following Equation 2, and
(3) a sieve mesh size and a wire diameter of a mesh included in the capillary rheometer satisfy the following Equation 3:
$$0.05 \leq \frac{{M}_{2}}{{M}_{1}} \leq 0.2 \qquad [ Equation 2 ]$$
wherein M₁ is a piston diameter, and M₂ is a capillary diameter,
$$0.65 \leq \frac{WD}{SM} \leq 0.73 \qquad [ Equation 3 ]$$
wherein WD is a wire diameter (mm), and SM is a sieve mesh size (mm),
$$\frac{ln ( \frac{{P}_{L}}{{P}_{0}} )}{V} < 0.16 \qquad [ Equation 5 ]$$
wherein P₀ is an initial internal pressure of the capillary rheometer when the PCR resin is added and a pressure was applied at a volume of 1 cc, P_{L} is a pressure finally applied to the capillary rheometer, and V is an internal volume of the capillary rheometer decreased by additional pressure from P₀, refers to a volume until a P_{L} value becomes twice a P₀ value, is measured with P_{L} being limited to twice P₀ when the P_{L} value is more than twice the P₀ value, and is set as a final measured volume when P_{L} is less than twice P₀, and
b) determining whether the PCR resin passing through the capillary rheometer satisfies each of Equations 5, 2 and 3, and if so then the PCR resin is an easily extrudable post-consumer recycled (PCR) resin.

9. The method for determining whether a post-consumer recycled (PCR) resin is an easily extrudable PCR resin of claim 8, wherein the PCR resin comprises a new material.

10. The method for determining whether a post-consumer recycled (PCR) resin is an easily extrudable PCR resin of claim 8, wherein the mesh comprised in the capillary rheometer is 50 to 200 meshes.

11. The method for determining whether a post-consumer recycled (PCR) resin is an easily extrudable PCR resin of claim 8, wherein the mesh has a total permeation area ratio of 35 to 50%.

12. The method for determining whether a post-consumer recycled (PCR) resin is an easily extrudable PCR resin of claim 8, wherein a cylinder internal temperature of the capillary rheometer satisfies the following Equation 4:
$${PT}_{1} + 50 ° C . < {T}_{1} < {PT}_{1} + 150 ° C . \qquad [ Equation 4 ]$$
wherein PT₁ is a melting temperature of the PCR resin, and T₁ is the cylinder internal temperature of the capillary rheometer.

13. The method for determining whether a post-consumer recycled (PCR) resin is an easily extrudable PCR resin of claim 8, wherein the capillary rheometer has the piston diameter of 10 to 30 mm and the capillary diameter of 1 to 5 mm.

14. An easily melt-extrudable post-consumer recycled (PCR) resin,
wherein a total capacity of a resin at which an initial pressure (P₀) becomes doubled (Pₜ) when the PCR resin passes through a capillary rheometer satisfies the following Equation 1,
a ratio between a piston diameter and a capillary diameter in the capillary rheometer satisfies the following Equation 2, and
a sieve mesh size and a wire diameter of a mesh included in the capillary rheometer satisfy the following Equation 3:
$$\frac{( {V}_{t} - {V}_{a} )}{{V}_{t}} < 0.8 \qquad [ Equation 1 ]$$
wherein Vₜ is an initial volume (cm³) of the PCR resin entering the capillary rheometer, and Vₐ is a volume (cm³) of a resin filtered by the mesh in the capillary rheometer at which the initial pressure becomes doubled,
$$0.05 \leq \frac{{M}_{2}}{{M}_{1}} \leq 0.2 \qquad [ Equation 2 ]$$
wherein M₁ is a piston diameter, and M₂ is a capillary diameter,
$$0.65 \leq \frac{W D}{S M} \leq 0.73 \qquad [ Equation 3 ]$$
wherein WD is a wire diameter (mm), and SM is a sieve mesh size (mm).

15. An easily extrudable post-consumer recycled (PCR) resin composition,
wherein the PCR resin comprises a polyolefin resin,
the following Equation 5 is satisfied when the PCR resin passes through a capillary rheometer,
a ratio between a piston diameter and a capillary diameter in the capillary rheometer satisfies the following Equation 2, and
a sieve mesh size and a wire diameter of a mesh included in the capillary rheometer satisfy the following Equation 3:
$$0.05 \leq \frac{{M}_{2}}{{M}_{1}} \leq 0.2 \qquad [ Equation 2 ]$$
wherein M₁ is a barrel diameter, and M₂ is a capillary diameter,
$$0.65 \leq \frac{W D}{S M} \leq 0.73 \qquad [ Equation 3 ]$$
wherein WD is a wire diameter (mm), and SM is a sieve mesh size (mm),
$$\frac{ln ( \frac{{P}_{L}}{{P}_{0}} )}{V} < 0.16 \qquad [ Equation 5 ]$$
wherein P₀ is an initial internal pressure of the capillary rheometer when the PCR resin is added and a pressure was applied at a volume of 1 cc, P_{L} is a pressure finally applied to the capillary rheometer, and V is an internal volume of the capillary rheometer decreased by additional pressure from P₀, refers to a volume until a P_{L} value becomes twice a P₀ value, is measured with P_{L} being limited to twice P₀ when the P_{L} value is more than twice the P₀ value, and is set as a final measured volume when P_{L} is less than twice P₀.

16. The easily extrudable PCR resin composition of claim 15, wherein the PCR resin composition comprises a new material.
